(19) United States
(12) Patent Application Publication
(10) Pub. No.: US 2022/0313663 A1
(43) Pub. Date: Oct. 6, 2022

(54) ELTROMBOPAG DOSING REGIMEN

(71) Applicant: NOVARTIS PHARMACEUTICALS CORPORATION, East Hanover, NJ (US)

(72) Inventors: Nichola COOPER, London (GB); Waleed GHANIMA, Gralum (NO); Miona STANKOVIC, Basel (CH); Francesco ZAJA, Udine (IT)

(21) Appl. No.: 17/611,012

(22) PCT Filed: May 14, 2019
(86) PCT No.: PCT/US2019/032171

§ 371 Date: Nov. 12, 2021

(51) Int. Cl. A61K 31/4152; A61P 7/04; A61K 31/573

(52) U.S. Cl. CPC A61K 31/4152; A61P 7/04; A61K 31/573

(57) ABSTRACT

The present invention relates to a method of treating immune thrombocytopenia (ITP) by administering eltrombopag or a pharmaceutically acceptable salt thereof to a patient.

### FIELD OF THE INVENTION

The present invention relates to a method of treating immune thrombocytopenia (ITP), especially chronic ITP, by administering eltrombopag or a pharmaceutically acceptable salt thereof to a patient. In particular, the method relates to the discontinuation of administration of eltrombopag or a pharmaceutically acceptable salt thereof while remaining a treatment-free remission after eltrombopag discontinuation. The method further relates to administering eltrombopag at a tapering regimen while remaining a remission.

### BACKGROUND OF THE INVENTION

Platelets (thrombocytes) are necessary for blood clotting. When platelet numbers are very low a patient is at risk of bleeding, hemorrhage, or death from hemorrhage. ITP is an autoimmune disorder characterized by transient or persistent decrease in platelet count with varied degree of bleeding. Eltrombopag is a highly effective treatment of ITP. It is used for long-term, even lifelong treatment. Long-term treatment with eltrombopag impends substantial costs and is associated with tolerability and compliance issues.

Recently, there have been a few reports on repeated short-term use [e.g. Bussel 2013, Br. J. Haematol.], tapering off [e.g. Bussel 2015, Blood], or discontinuation [e.g. Ghadaki 2013, Transfusion; Mahévas 2014, Br. J. Haematol.; C̆ervinek 2015, Int. J. Haematol.] of eltrombopag in ITP patients.

However, it is unknown what conditions need to be fulfilled for treatment-free remission after discontinuation of eltrombopag: what is the optimal stage to start discontinuation, modality of discontinuation, as well as the rate and duration of remission after discontinuation are still unclear and unsolved. It would be advantageous to provide a method for successful treatment of ITP with eltrombopag or a pharmaceutically acceptable salt thereof and subsequent discontinuation while maintaining treatment-free remission.

### SUMMARY OF THE INVENTION

The invention relates to a method of treating immune thrombocytopenia (ITP) comprising administering eltrombopag or a pharmaceutically acceptable salt thereof to a patient in need thereof, wherein
a) eltrombopag or a pharmaceutically acceptable salt thereof is administered at an initial dose until a complete response is achieved,
b) optionally eltrombopag or a pharmaceutically acceptable salt thereof is administered at the same dose as the initial dose during a consolidation phase,
c) optionally eltrombopag or a pharmaceutically acceptable salt thereof is administered according to a tapering regimen, and
d) administration of eltrombopag or a pharmaceutically acceptable salt thereof is discontinued.

### DETAILED DESCRIPTION OF THE INVENTION

### Introduction

The present invention provides a method of treating immune thrombocytopenia (ITP) comprising administering eltrombopag or a pharmaceutically acceptable salt thereof to a patient in need thereof, comprising
a. administering eltrombopag or a pharmaceutically acceptable salt thereof at an initial dose until a complete response is achieved,
b. optionally continuing to administer eltrombopag or a pharmaceutically acceptable salt thereof at the initial dose during a consolidation phase,
c. optionally administering a lower dose of eltrombopag or a pharmaceutically acceptable salt thereof according to a tapering regimen, and
d. discontinuing administration of eltrombopag or a pharmaceutically acceptable salt, wherein the patient achieves a treatment-free remission after discontinued administration.

Alternatively the present invention provides eltrombopag or a pharmaceutically acceptable salt thereof for use in the treatment of ITP comprising administering eltrombopag or a pharmaceutically acceptable salt thereof to a patient in need thereof, comprising
a. administering eltrombopag or a pharmaceutically acceptable salt thereof at an initial dose until a complete response is achieved,
b. optionally continuing to administer eltrombopag or a pharmaceutically acceptable salt thereof at the initial dose during a consolidation phase,
c. optionally administering a lower dose of eltrombopag or a pharmaceutically acceptable salt thereof according to a tapering regimen, and
d. discontinuing administration of eltrombopag or a pharmaceutically acceptable salt, wherein the patient achieves a treatment-free remission after discontinued administration.

In one embodiment of the present invention ITP refers to chronic ITP.

In another embodiment of the present invention eltrombopag or a pharmaceutically acceptable salt is present in the form of its bis-monoethanolamine (bis-olamine) salt.

In another embodiment of the present invention the initial dose is 100 mg, 75 mg, 50 mg, or 25 mg daily, suitably 50 mg daily.

In another embodiment of the present invention a complete response is achieved when the platelet count of said patient is at least 50,000/mL, at least 60,000/mL, at least 70,000/mL, at least 80,000/mL, at least 90,000/mL, or at least 100,000/mL, suitably at least 100,000/mL. Typically patient achieved complete response is in the absence of bleeding as well.

In another embodiment of the present invention the consolidation phase is at least one month, one month, at least two months, or two months, suitably two months.

In another embodiment of the present invention during the consolidation phase the platelet count of said patient is at least 50,000/mL, at least 60,000/mL, at least 70,000/mL, or at least 80,000/mL, or at least 100,000/mL, suitably at least 70,000/mL. Typically patient achieved complete response is in the absence of bleeding as well.

In another embodiment of the present invention the tapering regimen comprises one or more tapering doses, wherein there is a decrement between two adjacent tapering doses.

In another embodiment of the present invention the tapering regimen comprises one, two, three, or four tapering doses.

In another embodiment of the present invention there is a 50% decrement between two adjacent tapering doses.

In another embodiment of the present invention there is a 50% decrement between the first tapering dose and the second tapering dose.

In another embodiment of the present invention there is a 50% percent decrement between the second tapering dose and the third tapering dose.

In another embodiment of the present invention there is a 50% percent decrement between the third tapering dose and the fourth tapering dose.

In another embodiment of the present invention there is a 25 mg decrement between two adjacent tapering doses.

In another embodiment of the present invention there is a 12.5 mg decrement between two adjacent tapering doses for patients with Asian ancestry, wherein each tapering dose is administered for a period of two weeks.

In another embodiment of the present invention there is a 25 mg decrement between the first tapering dose and the second tapering dose.

In another embodiment of the present invention the last tapering dose of the tapering regimen is 25 mg daily, 12.5 mg daily, or 25 mg every other day.

In another embodiment of the present invention the initial dose is 100 mg daily, the first tapering dose is 75 mg daily, the second tapering dose is 50 mg daily, the third tapering dose is 25 mg daily, and optionally the fourth tapering dose is either 12.5 mg daily or 25 mg every other day.

In a particular embodiment of the present invention the initial dose is 75 mg daily, the first tapering dose is 50 mg daily, the second tapering dose is 25 mg daily, and optionally the third tapering dose is either 12.5 mg daily or 25 mg every other day.

In another particular embodiment of the present invention the initial dose is 50 mg daily, the first tapering dose is 25 mg daily, and the second i tapering dose is either 12.5 mg daily or 25 mg every other day.

In another embodiment of the present invention the initial dose is 25 mg daily and the tapering dose is either 12.5 mg daily or 25 mg every other day.

In another embodiment of the present invention each tapering dose is administered for a period of one week, two weeks, three weeks, or four weeks, suitably two weeks. Said period can be of different lengths for each tapering dose.

In another embodiment of the present invention during the tapering regimen the platelet count of said patient is at least 20,000/mL, at least 30,000/mL, at least 40,000/mL, or at least 50,000/mL, suitably at least 30,000/mL. Typically patient achieved complete response is in the absence of bleeding as well.

In another embodiment of the present invention after discontinuation of the administration of eltrombopag or a pharmaceutically acceptable salt thereof treatment-free remission is achieved.

In another embodiment of the present invention treatment-free remission is achieved when the platelet count of said patient is at least 20,000/mL, at least 30,000/mL, at least 40,000/mL, or at least 50,000/mL, suitably at least 30,000/mL. Typically patient achieved complete response is in the absence of bleeding as well.

In another embodiment of the present invention treatment-free remission is maintained for at least 1 month, at least 3 months, at least 6 months, at least 1 year, between 1 and 12 months, between 3 and 12 months, between 6 and 12 months, between 12 and 18 months, or between 12 and 24 months.

In another embodiment of the present invention treatment-free remission is achieved when a platelet count of at least 30,000/mL is maintained in the absence of bleeding, i.e. no bleeding adverse event, or in the absence of use of any rescue therapy, e.g. intravenous immunoglobulin.

In another embodiment of the present invention a treatment of ITP with eltrombopag or a pharmaceutically acceptable salt thereof can be resumed at least 1 month, at least 3 months, at least 6 months, or at least 1 year after the treatment-free remission status is lost.

In another embodiment of the present invention the resumption of treatment comprises the step of administering eltrombopag or a pharmaceutically acceptable salt thereof at the same dose as the initial dose.

In another embodiment of the present invention the resumption of treatment comprises the step of administering eltrombopag or a pharmaceutically acceptable salt thereof at a dose that is lower than the initial dose. “Lower than the initial dose” is understood here as either eltrombopag or a pharmaceutically acceptable salt thereof being administered at a lower dose per administration, or the interval between each administration being longer while the dose unchanged, or both.

In another embodiment of the present invention the resumption of treatment comprises the step of administering eltrombopag or a pharmaceutically acceptable salt thereof at a dose at least 25% or at least 50% lower than the initial dose.

In another embodiment of the present invention the resumption of treatment comprises the step of administering eltrombopag or a pharmaceutically acceptable salt thereof at a dose 25 mg less than the initial dose.

In another embodiment of the present invention the resumption of treatment comprises the step of administering eltrombopag or a pharmaceutically acceptable salt thereof every other day.

In another embodiment of the present invention the resumption of treatment comprises the step of administering eltrombopag or a pharmaceutically acceptable salt thereof at a dose of 25 mg daily.

In another embodiment of the present invention the resumption of treatment comprises the step of administering eltrombopag or a pharmaceutically acceptable salt thereof at a dose of 12.5 mg daily, especially for patients who are of East Asian ancestry.

In another embodiment of the present invention eltrombopag or a pharmaceutically acceptable salt thereof can be administered in combination with steroids.

In another embodiment of the present invention eltrombopag can be administered in combination with dexamethasone.

In another embodiment of the present invention eltrombopag or a pharmaceutically acceptable salt thereof is administered as a first line treatment.

In another embodiment of the present invention eltrombopag or a pharmaceutically acceptable salt thereof is administered as a second line treatment.

In another embodiment of the present invention eltrombopag or a pharmaceutically acceptable salt thereof is administered to patients who are ITP naive patient.

In another embodiment of the present invention eltrombopag or a pharmaceutically acceptable salt thereof is administered to patients who are refractory or relapsed after first-line steroids.

In another embodiment of the present invention eltrombopag or a pharmaceutically acceptable salt thereof is administered to an ITP patient whose platelet count is less than 30,000/mL.

In another embodiment of the present invention eltrombopag or a pharmaceutically acceptable salt thereof is administered to patients who relapsed following an initial response to a first-line course of steroid therapy, i.e. steroid-relapsed or steroid-refractory, with or without intravenous immunoglobulin used as a rescue therapy. In this embodiment, first line steroid therapy is defined as: prednisone/prednisolone 0.5 to 1 mg/kg/day for a minimum of 2 weeks, or minimum of 1 course of high-dose dexamethasone 20-40 mg/day for consecutive 4 days with or without intravenous immunoglobulin (used as rescue therapy), wherein maximum exposure to high dose steroids treatment (steroids tapering time excluded) is limited to 4 weeks of high dose prednisone/prednisolone or 3 courses of high-dose dexamethasone, wherein overall exposure to steroids less than 3 months, including the period of dose tapering.

In another embodiment of the present invention eltrombopag is administered to newly diagnosed primary ITP patients, wherein the time from diagnosis is within 3 months, with a platelet count less than 30,000/mL and a need for treatment, wherein optionally patients were treated with intravenous immunoglobulin 14 days before eltrombopag administration or with any ITP-directed therapy 3 days before eltrombopag administration.

In one embodiment In one embodiment newly diagnosed primary ITP patients start the treatment consisting of dexamethasone 40 mg/day for 4 consecutive days (days 1-4) and eltrombopag at a starting dose of 50 mg QD beginning at day 1.

In one further embodiment, after initiating eltrombopag at day 1, the dose must be adjusted by 25 mg increments to a maximum dose of 75 mg QD if the platelet count remains <50,000/mL following 2 weeks of therapy.

In one embodiment, eltrombopag treatment is continued with the minimal dosage necessary to achieve and maintain a platelet count≥50,000/mL for the duration of the consolidation phase. In one embodiment the consolidation phase is about 20 to 22 weeks long.

In one embodiment, the consolidation phase is followed by a tapering phase. In one embodiment the tapering phase is about 4 to 6 weeks.

In one embodiment the decrease in dose during the tapering phase will be performed by 25 mg reductions every 2 weeks, meaning that a next reduction will be carried out within 2 weeks until treatment discontinuation.

In another embodiment of the present invention eltrombopag in combination with dexamethasone induces a treatment-free response.

In another embodiment of the present invention eltrombopag induces a complete response at least once by week 4.

In another embodiment of the present invention eltrombopag increases the platelet count from baseline for 1, 2, 4, 12, 26 and 52 weeks.

### Definitions

As used herein, the terms “a” and “an” and “the” and similar references in the context of describing the invention are to be construed to cover both the singular and the plural, unless otherwise indicated herein or clearly contradicted by context. Where the plural form is used for compounds, salts, and the like, this is taken to mean also a single compound, salt, or the like.

The term or is used herein to mean, and is used interchangeably with, the term “and/or”, unless context clearly indicates otherwise.

Numerical values stated herein are to be understood as approximate values. This generally includes an acceptable degree of error for the value measured, due to the nature or precision of the measurements, and/or any underlying biological/chemical/physical fluctuations. Exemplary degrees of error are within 20%, typically, within 10%, and more typically, within 5% of a given value.

When describing a platelet count herein as a specified value, the actual value can vary by up to 20% from the stated amount. This recognizes that first, the precise amount of a measured platelet count may differ from the actual platelet number in the blood stream due to technical limitations of the method of measuring, and second, it recognizes that the platelet count can vary, fluctuate, or oscillate in a patient for various reasons and individual outlying measurements should not affect the overall assessment of a platelet count and conclusions drawn from it.

The terms “comprising” and “including” are used herein in their open-ended and non-limiting sense unless otherwise noted.

Platelet count may vary, fluctuate, or oscillate between measurements or can be imprecise due to limitations of the measuring methods. Therefore, a 20% variation, a 10% variation, or a 5% variation from any platelet count value stated herein should be tolerated. The frequency of the measurements can be adjusted by the treating physician. In some embodiments the platelet count is measured once per day, every other day, once per week, once every other week, once per month, once every other month, or once every 6 months. Suitably, the platelet count is first measured weekly, and once a stable platelet count is reached, the platelet count is measured monthly. The platelet count can be measured prior to initiation of the treatment with eltrombopag, throughout the treatment including a tapering regimen, and following discontinuation of treatment with eltrombopag. During adjustment of the initial dose of eltrombopag, platelet count might have to be measured more frequently than then following establishment of a stable initial dose of eltrombopag.

“Complete response” or “complete remission” is achieved when a platelet count reaches a defined level. The defined level is typically understood when the platelet count is at least 70,000/mL, at least 80,000/mL, at least 90,000/mL, at least 100,000/mL, at least 110,000/mL, or at least double or at least triple the baseline platelet count of the same patient. In a particular embodiment the defined level is at least 100,000/mL.

The term “consolidation phase” refers to the time period during which the platelet count is maintained above a defined value, which can be lower than that defined level for a complete response. In some embodiments said level is least 50,000/mL, at least 60,000/mL, at least 70,000/mL, or at least 80,000/mL. In a particular embodiment said level is at least 70,000/mL. In some embodiments the consolidation phase is at least one month, one month, at least two months, or two months. In a particular embodiment the consolidation phase is two months.

The term “tapering regimen” refers to a dosing regimen consisting of one, two, three or four tapering d doses, wherein the tapering dose is reduced stepwise, i.e. each dose is lower than its preceding dose. There can be one, two, thee, or four steps of dose reductions. In some embodiments each tapering dose is given during a period of one week, two weeks, three weeks, or four weeks. In another embodiment, each tapering dose is given for a different duration, e.g. the first tapering dose is given for 2 weeks and the second one for 4 weeks. In a particular embodiment a tapering dose is given during a period of two weeks. The dose decrement can be a certain percentage, e.g. 50%, of the preceding dose, or it can be an absolute decrement, e.g. 25 mg, of the preceding dose. During the tapering regimen phase, the platelet count is maintained at least 20,000/mL, at least 30,000/mL, at least 40,000/mL, or at least 50,000/mL during the tapering regimen. In a particular embodiment the platelet count is at least 30,000/mL during the tapering regimen.

The term “dose” refers to a certain amount of eltrombopag given at a certain frequency, e.g. 100 mg daily, 75 mg daily, 50 mg daily, 25 mg daily, or 12.5 mg daily. If an amount is not commercially available, it is possible to use a different amount and adjust the frequency to reach the same total dose, e.g. instead of 12.5 mg daily, the dose can be 25 mg every other day. If not otherwise specified, doses are daily doses.

The term “initial dose” refers to the dose the patient is given to achieve a complete response. A particular initial dose is 50 mg daily. A reduced dose of 25 mg daily is particular for patients who are of East Asian ancestry, e.g. Chinese, Japanese, Taiwanese, or Korean. The initial dose can be changed according to the response of the patient. The dose can be increased, e.g. to 75 mg or 100 mg, if the response is too weak, e.g. platelet count decreases, does not increase, or increases too slowly. The dose can be reduced, e.g. to 25 mg, if the response is too strong, e.g. platelets increase too quickly. The initial dose can be adjusted one or multiple times to reach the optimal initial dose for a patient. The initial dose is the dose that is administered before initiating a tapering regimen.

The term “tapering dose” refers to a dose that is administered after the patient had been treated with an initial dose. A tapering dose is lower than an initial dose.

The terms “discontinue” and “discontinuation” refer to the end of the treatment. The drug is not given to the patient anymore during a period of time. This period can either be a drug holiday, after which the drug administration is resumed, or it can be a final discontinuation. If drug administration is resumed the dose can be the same as the previous initial dose or less.

The term “treatment-free remission” refers to a remission that remains after discontinuation of the administration of eltrombopag or a pharmaceutically acceptable salt thereof. The platelet count of a patient during treatment-free remission is at least 20,000/mL, at least 30,000/mL, at least 40,000/mL, or at least 50,000/mL, suitably at least 30,000/mL.

Loss of remission response is achieved when the platelet count drops below 30,000/mL and/or a bleeding event occurs for a period of at least 2 weeks or at least one month.

### EXAMPLE

### Example 1: A Phase II, Open-Label, Prospective, Single-Arm, Study to Assess Ability of Eltrombopag to Induce Sustained Remission in Subjects with ITP Who Are Refractory or Relapsed After First-Line Steroids (TAPER)

### Purpose

The purpose of this trial is to assess the ability of eltrombopag to induce sustained treatment-free remission in ITP subjects who failed to respond or have relapsed after an initial treatment with steroids. There is limited, mainly retrospective evidence that earlier use of eltrombopag after ITP diagnosis, will allow a larger proportion of subjects to achieve sustained remission after tapering off drug. Clinically there is a need for a less toxic regimen that will provide responses and sustained remission with a shorter treatment interval. This trial is designed to assess this.

### Objectives

The primary objective is to assess the ability of eltrombopag to induce sustained remission by month 12 in ITP subjects who relapsed or failed to respond to first-line steroid treatment. The primary endpoint is the proportion of subjects with sustained remission by month 12, where sustained remission is defined as:
reach a platelet count≥100,000/mL (complete response)
maintain a platelet count of around 100,000/mL for 2 months (meaning no platelet count below 70,000/mL)
taper off drug until treatment discontinuation
maintain a platelet count≥30,000/mL in the absence of bleeding (no bleeding adverse event) or use of any rescue therapy until month 12.

The main secondary objectives are:
To assess the duration of sustained remission after treatment discontinuation. Endpoint: Median duration of sustained remission (weeks) counted from last dose of eltrombopag to month 12 for subjects with sustained remission.
To assess the ability of eltrombopag to induce early response by month 1. Endpoint: Number (%) of subjects with platelet count≥50,000/mL at least once by month 1 (first month) without bleeding and no rescue therapy.
To assess the ability of eltrombopag to induce a recovery response, in case of loss of response during or after tapering of eltrombopag. Endpoint: Number (%) of subjects with at least one platelet count≥30,000/mL after eltrombopag is re-introduced, in case of loss of response (<30,000/mL and/or bleeding event) without bleeding and no rescue therapy.
To quantify the platelet count from baseline to 3, 6, 9, 12 months. Endpoint: Absolute and relative change in platelet count from baseline to 3, 6, 9,12 months and end-of-study.
To assess the ability of eltrombopag to maintain platelet count≥30,000/ml within 12 months. Endpoint: Number (%) of subjects who maintain a platelet count≥30,000/mL from the first time of reaching that level to month 3, 6, 9, 12 and end-of-study without bleeding and no rescue therapy.

### Population

The study is designed to include adult subjects with primary ITP who have failed to respond or who relapsed following an initial response to a first-line course of steroid therapy (steroid-relapsed or steroid-refractory) with or without ±intravenous immunoglobulin (IVIG) used as a rescue therapy. Platelet count is <30,000/mL and assessed as needing treatment (per physician's discretion). First line of steroid therapy will be defined as: prednisone/prednisolone 0.5 to 1 mg/kg/day for a minimum of 2 weeks, or minimum of 1 course of high-dose dexamethasone 20-40 mg/day for consecutive 4 days ±IVIG (used as rescue therapy). Maximum exposure to high dose steroids treatment (steroids tapering time excluded) should be limited to: 4 weeks of high dose prednisone/prednisolone or 3 courses of high-dose dexamethasone. Overall exposure to steroids must not be longer than 3 months, including period of dose tapering. Main exclusion criteria are:
1. ITP subjects previously treated with any ITP second-line therapies, thrombopoietin receptor agonists for ITP, except steroids/IVIG.
2. Subjects who relapsed more than one year after the end of first-line full course of steroid therapy.
3. Subjects with a diagnosis of secondary thrombocytopenia.
4. Subjects who have life threatening bleeding complications per investigator discretion.

### Treatment

Subjects will be treated with eltrombopag 50 mg daily (QD) for 2 weeks to reach a target platelet count of 100,000/mL. For those subjects who do not achieve the target platelet count within 2 weeks, the dose of eltrombopag will be increased to 75 mg QD. Those subjects who have not reached the target platelet count 100,000/mL after being treated with eltrombopag 75 mg/day will continue on eltrombopag until month 12. Treatment with eltrombopag will be continued at the minimal dosage necessary to achieve and maintain a platelet count around 100,000/mL for 2 months.

Subjects will be eligible for taper off and treatment discontinuation if they have a reached a complete response (CR), defined as platelet counts≥100,000/mL, and maintained platelet counts around 100,000/mL for 2 months (no counts below 70,000/mL).

The duration of tapering will be individualized and depend upon starting dose and response of the subject: decreases in dose will be performed by 25 mg reductions every 2 weeks. If platelet counts are stable, the next reduction should be carried out within 2 weeks, with dosing at 25 mg on alternate days for 2 weeks until treatment is totally discontinued.

A reduced initial dosage of 25 mg QD is recommended for subjects of Asian ancestry. The dose of eltrombopag in subjects with Asian ancestry will be reduced to account for the lower eltrombopag clearance (CL/F) observed in these subjects compared to the non-Asian population. Therefore, drug tapering for subjects with Asian ancestry will be done in 12.5 mg decrements every second week.

Subjects who successfully taper off, i.e. discontinue eltrombopag and maintain platelet count≥30,000/mL in the absence of bleeding or use of any rescue therapy will be followed up through the duration of the trial (to month 12). In case of relapse, subjects will be offered a new course of eltrombopag treatment within this timeframe, starting at a dose of 50 mg.

Subjects with bleeding, or perceived risk of serious bleeding, will be allowed to receive rescue therapy as per physician decision, irrespective of platelet counts. Platelet count will be performed at screening visit 1 to assess the eligibility of the subject.

Hematology including platelet counts will be assessed at week 1/day 1 and weekly during the first 8 weeks of treatment. Based on subject response, hematology will be performed biweekly thereafter until the end of treatment. Additional assessments of platelet count may be performed more frequently than the biweekly schedule if needed in accordance with the clinical judgment of the investigator. Bleeding events will be assessed at each visit.

### Example 2: TAPER II

### Purpose

The purpose of this trial is to assess the ability of eltrombopag to induce sustained treatment-free remission in ITP subjects who failed to respond or have relapsed after an initial treatment with steroids and who receive treatment with eltrombopag.

### Objectives

The primary objective is to assess the ability of eltrombopag to induce sustained response off treatment at month 6 in ITP subjects who were treated with second line eltrombopag and previously achieved CR. The primary endpoint is the proportion of subjects with sustained response off treatment at month 6 wherein sustained response off treatment is defined as:
reach platelet count≥100,000/mL (complete response) and then maintain platelet counts around 100,000/mL for 2 months (no counts below 70,000/mL) and then
taper off the drug until treatment discontinuation while,
maintain platelet count≥30,000/mL in the absence of bleeding (no bleeding adverse events) or use of any rescue therapy until month 6.

### Population

The study is designed to include adult subjects with ITP who failed to respond or who relapsed after first-line steroid treatment and are on treatment with eltrombopag. Platelet count is ≥100,000/mL and stable (above 70,000/mL) for 2 months.

### Treatment

Subjects will begin stepwise reduction of the eltrombopag dose on day 1. In case of relapse they will be re-treated with eltrombopag. Relapse is defined as platelet count<30,000/mL. Retreatment will be with 50 mg-75 mg eltrombopag until end-of-study. Subjects in sustained response off treatment at month 6 will enter a 1 year follow-up period. Sustained response off treatment is defined as:
Reach platelet count of 100,000/mL (complete response) and then maintain platelet counts about 100,000/mL for 2 months (no counts below 70,000/mL) and
then taper off the drug until treatment discontinuation while,
maintain platelet count≥30,000/mL in the absence of bleeding (no bleeding adverse events) or use of any rescue therapy until month 6.

End-of-study is after 1.5 years.

Optionally, the study includes a second arm which are patients on second line steroids. The steroids will be stopped on day 1 and there will be re-treatment with steroids or eltrombopag after relapse. The timing and endpoints of the second arm are the same and subjects in sustained response off treatment at month 6 will also enter a 1 year follow-up period.

### Example 3: A Phase II, Randomized (1:1) Open Label Study to Assess the Efficacy and Safety of Eltrombopag in Combination with Dexamethasone Compared to Dexamethasone, as First-Line Treatment in Adult Patients with Newly Diagnosed Immune Thrombocytopenia

### Purpose

The purpose of this trial is to assess if intensification of first-line therapy can induce superior long-term remission rates. Spontaneous remissions occur in about 10% of adult patients.

Traditional first-line agents achieve high initial response rates (70-80%) within 2 to 14 days, but have suboptimal durability. Sustained platelet responses after discontinuation of corticosteroid occurs in <30% of patients within the first year. Approximately 50% of patients have relapsed by 6 months, with an additional 25% relapsing beyond 1 year. Long-term administration of corticosteroids is limited by the development of side effects.

Therefore, there is an unmet medical need for a less toxic regimen that will provide responses and sustained remission with a shorter treatment interval. The aim is to reduce chronic steroid use (high doses of steroids associated with morbidity) and to improve treatment-free remission.

### Population

This study is designed to include newly diagnosed adult primary ITP-patients (time from diagnosis within 3 months) with a platelet count 30,000/mL and a need for treatment. In case of clinical necessities because of very low platelet count and/or significant bleeding, treatment with intravenous immunoglobulin is permitted 14 days before randomization (alternative: any ITP-directed therapy 3 days before randomization might be permitted). Otherwise, previous history of treatment for ITP is an exclusion criterium.

### Objectives

The primary objective is to assess the ability of eltrombopag in combination with dexamethasone to induce a treatment-free response at week 52. Treatment-free response is defined as maintaining a platelet count≥30,000/mL after treatment discontinuation in the absence of bleeding≥Grade II or use of any rescue medication.

Main secondary objectives are:
Assess the duration of treatment-free response after treatment discontinuation. Endpoint: Median duration of treatment-free response calculated from the time of treatment discontinuation until platelet count<30,000/mL or bleeding Grade II or use of rescue therapy.
Assess the ability of eltrombopag in combination with dexamethasone to induce a treatment-free response at week 78. Endpoint: Rates of patients achieving a treatment free response at week 78.
Assess the ability of eltrombopag to induce overall response by week 4. Endpoint: Proportion of patients with platelet count≥30,000/mL and absence of bleeding and no rescue therapy at least once by week 4.
Assess the ability of eltrombopag to induce complete response by week 4. Endpoint: Proportion of patients with platelet count≥100,000/mL and absence of bleeding and no rescue therapy at least once by week 4.
Quantify the increase in platelet count from baseline to 1, 2, 4, 12, 26 and 52 weeks. Endpoint: Absolute and relative change in platelet count from baseline to 1, 2, 4, 12, 26 and 52 weeks.
Assess the time to response. Endpoint: Time from starting treatment to time of achievement of response.

### Treatment

Patients will be randomized to one of two treatment arms in a 1:1 ratio. Arm A is eltrombopag in combination with dexamethasone and Arm B is dexamethasone alone.

Arm A consists of treatment from day 1 until week 26 including tapering of eltrombopag. Tapering of eltrombopag is either 4 weeks after 22 weeks of induction therapy or 6 weeks after 20 weeks of induction therapy. The term “induction therapy” is understood as the total period during which eltrompobag or a pharmaceutically acceptable salt thereof is administered before the dose is reduced. Typically induction therapy includes the period during which patient are treated to achieve complete response and the following period during which patient are treated to maintain the remission (consolidation phase).

Treatment consists of dexamethasone 40 mg/day for 4 consecutive days (days 1-4) and eltrombopag at a starting dose of 50 mg QD beginning at day 1. After initiating eltrombopag at day 1, the dose must be adjusted by 25 mg increments to a maximum dose of 75 mg QD if the platelet count remains <50,000/mL following 2 weeks of therapy. Eltrombopag treatment is continued with the minimal dosage necessary to achieve and maintain a platelet count≥50,000/mL for the duration of the induction period (until week 20 or week 22) followed by a 4 week or 6 week taper to discontinue eltrombopag. During tapering of eltrombopag, a decrease in dose will be performed by 25 mg reductions every 2 weeks, meaning that a next reduction will be carried out within 2 weeks until treatment discontinuation.

Arm B consists of treatment from day 1 until week 12. The treatment consists of 1-3 cycles of pulsed dexamethasone 40 mg/day for 4 consecutive days (days 1-4) at 4-week intervals. There is no initiation of a next cycle with dexamethasone if platelet count is >150,000/mL. Subjects of both study arms at month 6 will enter a maximum 1 observational period in which they are observed for the primary endpoint. After that there is a follow-up at 78 weeks.

1. A method of treating immune thrombocytopenia (ITP) comprising administering eltrombopag or a pharmaceutically acceptable salt thereof to a patient in need thereof, comprising
a. administering eltrombopag or a pharmaceutically acceptable salt thereof at an initial dose until a complete response is achieved,
b. optionally continuing to administer eltrombopag or a pharmaceutically acceptable salt thereof at the initial dose during a consolidation phase,
c. optionally administering a lower dose of eltrombopag or a pharmaceutically acceptable salt thereof according to a tapering regimen, and
d. discontinuing administration of eltrombopag or a pharmaceutically acceptable salt, wherein the patient achieves a treatment-free remission after discontinued administration.

2. The method of claim 1, wherein the initial dose is 100 mg, 75 mg, 50 mg, or 25 mg daily.

3. The method of claim 1, wherein a complete response is achieved when the platelet count of said patient is at least 50,000/mL, at least 60,000/mL, at least 70,000/mL, at least 80,000/mL, at least 90,000/mL, or at least 100,000/mL, suitably at least 100,000/mL.

4. The method of claim 1 wherein the consolidation phase is at least one month, one month, at least two months, or two months.

5. The method of claim 1 wherein during the consolidation phase the platelet count of said patient is at least 50,000/mL, at least 60,000/mL, at least 70,000/mL, or at least 80,000/mL.

6. The method of claim 1 wherein the tapering regimen comprises one or more tapering doses, wherein there is a decrement between two adjacent tapering doses.

7. The method of claim 6 wherein the tapering regimen comprises one, two, three, or four tapering doses.

8. The method of claim 7 wherein there is a 50% decrement between two adjacent tapering doses.

9. The method of claim 8 wherein there is a 25 mg decrement between two adjacent tapering doses.

10. The method of claim 9 wherein the last tapering dose of the tapering regimen is 25 mg daily, 12.5 mg daily, or 25 mg every other day.

11. The method of claim 10 wherein each tapering dose is administered for a period of one week, two weeks, three weeks, or four weeks, suitably two weeks.

12. The method of claim 11 wherein during the tapering regimen the platelet count of said patient is at least 20,000/m L, at least 30,000/m L, at least 40,000/mL, or at least 50,000/m L, suitably at least 30,000/m L.

13. The method of claim 12 wherein treatment-free remission is achieved when the platelet count of said patient is at least 20,000/mL, at least 30,000/mL, at least 40,000/mL, or at least 50,000/mL, suitably at least 30,000/m L.

14. A method of treating immune thrombocytopenia (ITP) in a patient, especially who are refractory or relapsed after first-line steroids, comprising administering eltrombopag or a pharmaceutically acceptable salt thereof to a patient in need thereof, comprising
a. administering eltrombopag or a pharmaceutically acceptable salt thereof at an initial dose until a complete response is achieved,
b. continuing to administer eltrombopag or a pharmaceutically acceptable salt thereof at the initial dose during a consolidation phase for 2 months,
c. administering one, two or three tapering doses of eltrombopag or a pharmaceutically acceptable salt thereof, each one for 2 weeks, wherein the first tapering dose is 25 mg less than the initial dose except when the initial dose is 25 mg, wherein the last tapering dose is either 25 mg daily or 12.5 mg daily or 25 mg every other day; and
d. discontinuing administration of eltrombopag or a pharmaceutically acceptable salt, wherein the patient achieves a treatment-free remission after discontinued administration.
